(19) United States
(12) Patent Application Publication
(10) Pub. No.: US 2003/0004207 A1
(43) Pub. Date: Jan. 2, 2003

(54) USE OF COMPOUNDS WHICH ACTIVATE A 5-HT RECEPTOR TO TREAT URINARY INCONTINENCE

(76) Inventor: DOUGLAS A. CRAIG, EMERSON, NJ (US)

Correspondence Address: JOHN P WHITE, COOPER & DUNHAM LLP, 1185 AVENUE OF THE AMERICAS, NEW YORK, NY, 10036

(21) Appl. No.: 09/450,880

(22) Filed: Nov. 29, 1999

(51) Int. Cl. A61K 31/405

(52) U.S. Cl. 514/415; 514/19

(57) ABSTRACT

The invention provides a method of treating urinary incontinence in a subject which comprises administering to the subject a therapeutically effective amount of a 5-HT receptor agonist.

### BACKGROUND OF THE INVENTION

Throughout this application, various publications are referenced in parentheses by author and year. Full citations for these references may be found at the end of the specification immediately preceding the claims. The disclosures of these publications in their entireties are hereby incorporated by reference into this application to describe more fully the art to which this invention pertains.

Normal bladder function requires coordinated bladder contraction and urethral sphincter relaxation during the emptying phase and the opposite, i.e. bladder relaxation and urethral sphincter contraction, during the filling/storage phase. The occurrence of irregularities in these processes increases with age in males and females and can lead to a variety of disorders of lower urinary tract function, including incontinence and urinary retention.

Incontinence

Incontinence is characterized by the involuntary loss of urine. It can be categorized generally into two types: stress and urge incontinence. Stress incontinence is characterized by an inability to generate or maintain normal closing pressure at the bladder outlet and urethra. Involuntary urine loss can be provoked whenever the physical stress of everyday activities causes intra-abdominal pressure (which is translated directly to the bladder) to rise above the level of the bladder closing pressure; hence the name. The underlying pathology may involve (1) damage to the nerves which send contractile signals to the urethra and bladder outlet (such as may occur during childbirth), (2) a decrease in the thickness of the urothelium after menopause, or (3) other processes which result in primary muscle weakness or loss of tone to these structures (Wein, 1987; Andersson, 1988).

Urge incontinence, in contrast, involves primarily changes in the function of the main body (detrusor) of the bladder. These changes may arise as a result of bladder inflammation (as in interstitial cystitis), secondary to bladder outlet obstruction (as in benign prostatic hyperplasia), or may be idiopathic. These changes disrupt the normal manner in which bladder volume/distention is sensed and translated into, first, the desire to void and, ultimately, the neuronal impulses signaling detrusor contraction. Rather than the normal graded response to bladder distention, patients with urge incontinence experience involuntary leakage preceded by a sensation of urgency and impending urinary loss. A large portion of patients with urge incontinence are shown by urodynamic evaluation to have detrusor instability, characterized by spontaneous, involuntary contractions (Swami and Abrams, 1996). The underlying pathology may involve the sensory or motor nervous systems innervating the detrusor, may be associated with inflammation and irritative processes in the bladder muscle, may arise secondary to chronic outlet obstruction from conditions such as benign prostatic hyperplasia (BPH), or may be idiopathic (Andersson, 1988).

Neural Control of the Micturition Reflex

The micturition reflex is initiated by primary afferent Aδ and C type neurons which are activated in response to stretch of the bladder wall. These fibers pass via the pelvic nerve to the sacral spinal cord (DeGroat and Steers, 1990). Here they make synaptic connections with secondary afferent neurons which send excitatory signals to the pontine micturition center (PMC) in the brainstem. When activated, neurons of the PMC transmit excitatory signals back to the sacral spinal cord to inhibit sympathetic nerves responsible for maintaining tone to the bladder base and urethra, and to simulate parasympathetic motor nerves which contract the detrusor (Wein, 1987; DeGroat, 1998).

In addition to excitatory input from spinal neurons, the PMC receives various modulatory inputs from higher brain centers. Studies have shown that GABA (γ-aminobutyric acid) dopamine, acetylcholine, and enkephalins modulate neurotransmission in the PMC (DeGroat, 1998). In addition, serotonin (5-hydroxytryptamine, 5-HT), glycine, GABA and enkephalins also act at the level of the sacral spinal cord to influence micturition. Serotonergic projections from the raphe nuclei to the sacral spinal cord are thought to exist, since stimulation of these nerves inhibits bladder activity (DeGroat and Steers, 1990).

Current Treatments for Micturition Disorders

A variety of pharmaceutical agents has been employed to treat micturition disorders. Drugs used to reduce bladder contractility associated with urge incontinence include muscarinic receptor antagonists, calcium channel blockers, direct smooth muscle relaxants, and beta-adrenergic receptor agonists (Andersson, 1988). In addition, inhibitors of prostaglandin synthesis and tricylic antidepressants have been investigated for this purpose (Andersson, 1988). More recently, there has been an interest in developing potassium channel openers, aimed at reducing contractility by hyperpolarizing detrusor smooth muscle cells (Grant et al., 1994). Each of these therapies is associated with limited efficacy and unwanted side effects (Andersson, 1988; Wein, 1987). Furthermore, each of these therapies targets the motor component of micturition, and thereby has the potential for compromising bladder contraction required for the efficient elimination of urine.

Discovery of 5-HT_{1F} Receptor Role in the Control of Micturition

The investigations leading to the present discovery arose from our discovery that messenger RNA for the 5-HT_{1F} receptor is present in rat spinal cord (see U.S. Pat. No. 5,639,652). This discovery led us to hypothesize that the 5-HT_{1F} receptor may play a role in controlling the activity of afferent sensory neurons and, therefore, might be an important target for the control of the micturition reflex by 5-HT or by agents which act selectively at the 5-HT_{1F} receptor.

To explore this hypothesis, the effects of 5-HT_{1F} selective agents were investigated in an isovolumic model of micturition in the rat. This invention relates to the discovery that administration of selective 5-HT_{1F} agonists produces inhibition of distension-induced rhythmic contractions in this model, and that this effect is blocked by the prior administration of 5-HT_{1F}-receptor-selective antagonists. This model of the urinary reflex is considered by experts in the field of urology and lower urinary tract function to reflect the potential of agents to modulate micturition in humans (see Maggi et al., 1987; Morikawa et al., 1992; Yoshiyama et al., 1995; and PCT International Application No. WO 97/31637).

### SUMMARY OF THE INVENTION

The present invention provides a method of treating urinary incontinence in a subject which comprises administering to the subject a therapeutically effective amount of a 5-HT_{1F} receptor agonist which activates the human 5-HT_{1F} receptor at least ten-fold more than it activates each of the human 5-HT_{1A}, 5-HT_{1D}, 5-HT_{2A}, 5-HT_{2C}, 5-HT₃, 5-HT₄, and 5-HT₇ receptors.

Preferably, the aforementioned 5-HT_{1F} receptor agonist additionally activates the human 5-HT_{1F} receptor at least ten-fold more than it activates each of the human 5-HT_{1B}, 5-HT_{1E}, 5-HT_{2B}, 5-HT_{5A}, 5-HT_{5B}, and 5-HT₆ receptors.

In addition, the invention provides a process for making a composition of matter which specifically binds to a human 5-HT_{1F} receptor which comprises identifying a chemical compound using any of the processes described herein for identifying a compound which binds to and/or activates or inhibits activation of a human 5-HT_{1F} receptor and then synthesizing the chemical compound or a novel structural or functional analog or homolog thereof.

This invention further provides a process for preparing a pharmaceutical composition which comprises admixing a pharmaceutically acceptable carrier and a pharmaceutically acceptable amount of a chemical compound identified by any of the processes described herein for identifying a compound which binds to and/or activates or inhibits activation of a human 5-HT_{1F} receptor or a novel structural or functional analog or homolog thereof.

### BRIEF DESCRIPTION OF THE FIGURES

A more complete understanding of the invention and many of its advantages will become apparent by reference to the detailed description which follows when considered in conjunction with the accompanying figures, wherein:

FIG. 1 shows distension-induced rhythmic contractions of the urinary bladder in a urethane-anesthetized rat and inhibition of the contractions by the 5-HT_{1F}-selective agonist Compound 1. Ordinate, Bladder Pressure in mmHg; abscissa, time.

FIG. 2 shows distension-induced rhythmic contractions of the urinary bladder in a urethane-anesthetized rat and inhibition of the contractions by the 5-HT_{1F}-selective agonist Compound 2. Ordinate, Bladder Pressure in mmHg; abscissa, time.

FIG. 3 shows that the inhibitory effects of the 5-HT_{1F}-selective agonist Compound 2 are blocked by the prior administration of the 5-HT_{1F}-selective antagonist Compound 3. Specificity of the antagonism is demonstrated by the failure of Compound 3 to block the inhibitory effect of the GABA_{B} receptor agonist, 3-aminopropyl-methyl-phosphinic acid (3-APMPA), on bladder contractions. Ordinate, Bladder Pressure in mmHg; abscissa, time.

### DETAILED DESCRIPTION OF THE INVENTION

The following definitions are presented as an aid in understanding this invention.

Receptor Activation describes the process by which the binding of a compound to the receptor when it is on the surface of a cell leads to a metabolic response within the cell. Such metabolic responses include, but are not limited to, activation of adenylate cyclase, activation of guanylate cyclase, hydrolysis of inositol phospholipids, movement of ions across the cell membrane, or contraction in a tissue in the cells in which the receptor is expressed.

Potency means the concentration of an agonist which elicits half of its own maximum activation (expressed as EC₅₀ or the negative log of the EC₅₀, i.e., pEC₅₀)

Intrinsic Activity means the magnitude of the maximum activation in a cell or tissue which a particular agonist is capable of eliciting, relative to the maximum activation elicited by a reference full agonist, and expressed as values ranging between unity for full agonists (e.g., serotonin in the case of 5-HT receptors) and zero for antagonists. Because intrinsic activity as originally defined by Ariens (Ariens et al., 1960) is recognized as being dependent upon the receptor system in which it is measured (Kenakin, 1987), intrinsic activity as referred to in this document is based upon measurements made using the cloned human 5-HT receptor systems described below.

Selectivity of Receptor Activation refers to the ability of an agonist to selectively activate one receptor relative to another receptor. Such selectivity may reflect either (a) the agonist's ability to activate one receptor at a much lower concentration than required to activate another receptor (i.e., a potency difference) or (b) the agonist's ability to activate one receptor to a much greater degree than another receptor, independent of concentration (i.e., an intrinsic activity difference), or (c) a combination of both.

Therefore, statements of the form “activates the 5-HT_{1F} receptor ten-fold more than it activates one of the following (receptors)” mean and include any such difference whether it is by virtue of a difference in potency, or a difference in intrinsic activity, or both.

Having due regard to the preceding definitions, the present invention provides a method of treating urinary incontinence in a subject which comprises administering to the subject a therapeutically effective amount of a 5-HT_{1F} receptor agonist which activates the human 5-HT_{1F} receptor at least ten-fold more than it activates each of the human 5-HT_{1A}, 5-HT_{1D}, 5-HT_{2A}, 5-HT_{2C}, 5-HT₃, 5-HT₄, and 5-HT₇ receptors.

Preferably, the aforementioned 5-HT_{1F} receptor agonist additionally activates the human 5-HT_{F1} receptor at least ten-fold more than it activates each of the human 5-HT_{1B}, 5-HT_{1E}, 5-HT_{2B}, 5-HT_{5A}, 5-HT_{5B}, and 5-HT₆ receptors.

More preferably, the 5-HT_{1F} receptor agonist activates the human 5-HT_{1F} receptor at least 50-fold more than it activates each of the human 5-HT_{1A}, 5-HT_{1D}, 5-HT_{2A}, 5-HT_{2C}, 5-HT₃, 5-HT₄, and 5-HT₇ receptors.

Preferably, the aforementioned 5-HT_{1F} receptor agonist additionally activates the human 5-HT_{1F} receptor at least 50-fold more than it activates each of the human 5-HT_{1B}, 5-HT_{1E}, 5-HT_{2B}, 5-HT_{5A}, 5-HT_{5B}, and 5-HT₆ receptors.

Still more preferably, the 5-HT_{1F} receptor agonist activates the human 5-HT_{1F} receptor at least 100-fold more than it activates each of the human 5-HT_{1A}, 5-HT_{1D}, 5-HT_{2A}, 5-HT_{2C}, 5-HT₃, 5-HT₄, and 5-HT₇ receptors.

Preferably, the aforementioned 5-HT_{1F} receptor agonist additionally activates the human 5-HT_{1F} receptor at least 100-fold more than it activates each of the human 5-HT_{1B}, 5-HT_{1E}, 5-HT_{2B}, 5-HT_{5A}, 5-HT_{5B}, and 5-HT₆ receptors.

Most preferably, the 5-HT_{1F} receptor agonist activates the human 5-HT_{1F} receptor at least 200-fold more than it activates each of the human 5-HT_{1A}, 5-HT_{1D}, 5-HT_{2A}, 5-HT_{2C}, 5-HT₃, 5-HT₄, and 5-HT₇ receptors.

Preferably, the aforementioned 5-HT_{1F} receptor agonist additionally activates the human 5-HT_{1F} receptor at least 200-fold more than it activates each of the human 5-HT_{1B}, 5-HT_{1E}, 5-HT_{2B}, 5-HT_{5A}, 5-HT_{5B}, and 5-HT₆ receptors.

In certain embodiments the 5-HT_{1F} receptor agonist also activates the human 5-HT_{1F} receptor at least ten-fold more than it activates any human α₂ adrenoceptor or any human β adrenoceptor.

In other embodiments the 5-HT_{1F} receptor agonist also activates the human 5-HT_{1F} receptor at least ten-fold more than it activates the human histamine H₁ and H₂ receptors.

In still other embodiments the 5-HT_{1F} receptor agonist also activates the human 5-HT_{1F} receptor at least ten-fold more than it activates the human dopamine D₁, D₂, D₃, and D₅ receptors.

In still further embodiments the 5-HT_{1F} receptor agonist also activates the human 5-HT_{1F} receptor at least ten-fold more than it activates the human α_{1A} adrenoceptor and the human α_{1B} adrenoceptor.

In addition, the invention provides a process for making a composition of matter which specifically binds to a human 5-HT_{1F} receptor which comprises identifying a chemical compound using any of the processes described herein for identifying a compound which binds to and/or activates or inhibits activation of a human 5-HT_{1F} receptor and then synthesizing the chemical compound or a novel structural or functional analog or homolog thereof.

This invention further provides a process for preparing a pharmaceutical composition which comprises admixing a pharmaceutically acceptable carrier and a pharmaceutically acceptable amount of a chemical compound identified by any of the processes described herein for identifying a compound which binds to and/or activates or inhibits activation of a human 5-HT_{1F} receptor or a novel structural or functional analog or homolog thereof.

Without intending to be limited thereby, it is suggested that the mechanism by which the present invention treats urinary incontinence in a subject to whom a therapeutically effective amount of a 5-HT_{1F} receptor agonist is administered is by reducing afferent urinary impulses from sensory nerves in the bladder.

The binding and functional properties of compounds at the different human receptors were determined in vitro using cultured cell lines that selectively express the receptor of interest. These cell lines were prepared by transfecting the cloned cDNA or cloned genomic DNA or constructs containing both genomic DNA and cDNA encoding the human 5-HT receptors as further described in detail in the Experimental Details herein below.

In connection with this invention, a number of cloned human receptors discussed herein, as stably transfected cell lines, have been made pursuant to, and in satisfaction of, the Budapest Treaty on the International Recognition of the Deposit of Micro-organisms for the Purpose of Patent Procedure, and are made with the American Type Culture Collection, 10801 University Blvd., Manassas, Va. 20110-2209. Specifically, these deposits have been accorded ATCC Accession Numbers as follows:

ATCC Deposits:

| | | ATCC | |
|---|---|---|---|
| | | Accession | Date of |
| Designation | Receptor | No. | Deposit |
| Ltk-8-30-84* | human 5-HT_{1D} | CRL 10421 | Apr. 17, 1990 |
| Ltk-11* | human 5-HT_{1B} | CRL 10422 | Apr. 17, 1990 |
| 5HT_{1E}-7 | human 5-HT_{1E} | CRL 10913 | Nov. 06, 1991 |
| L-5-HT_{1F} | human 5-HT_{1F} | CRL 10957 | Dec. 27, 1991 |
| L-5HT-4B* | human 5-HT₇ | CRL 11166 | Oct. 20, 1992 |
| 5HT1A-3 | human 5-HT_{1A} | CRL 11889 | May 11, 1995 |
| L-NGC-5HT₂* | human 5-HT_{2A} | CRL 10287 | Oct. 31, 1989 |

*The “5-HT_{1D1}”, “5-HT_{1D2}”, “5-HT_{4B}”, and “5-HT₂” receptors were renamed the “5-HT_{1D}” , “5-HT_{1B}”, “5-HT₇”, and “5-HT_{2A}” receptors, respectively, by the Serotonin Receptor Nomenclature Committee of the IUPHAR.

The data shown in the accompanying Table and Figures indicate that activation of the 5-HT_{1F} serotonin receptor inhibits the micturition reflex. This in vivo property is recognized in the art as correlating with efficacy in treating urinary incontinence.

The present invention therefore provides a method of treating urinary incontinence, which comprises administering a quantity of any of the 5-HT_{1F} receptor agonists defined herein in a quantity effective against urinary incontinence. The drug may be administered to a patient afflicted with urinary incontinence by any conventional route of administration, including, but not limited to, intravenous, intramuscular, oral, subcutaneous, intratumoral, intradermal, and parenteral. The quantity effective against urinary incontinence is between about 0.001 mg and about 10.0 mg per kg of subject body weight.

The method of treating urinary incontinence disclosed in the present invention may also be carried out using a pharmaceutical composition comprising any of the 5-HT_{1F} receptor agonists as defined herein and a pharmaceutically acceptable carrier. The composition may contain between about 0.05 mg and about 500 mg of a 5-HT_{1F} receptor agonist, and may be constituted into any form suitable for the mode of administration selected. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The drug may otherwise be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings. The drug may also be formulated as a part of a pharmaceutically acceptable transdermal patch. Such transdermal patches may be used to provide continuous or discontinuous infusion of pharmaceutical agents in controlled amounts. The construction and use of transdermal patches for the delivery of pharmaceutical agents is well known in the art. (See, for e.g., U.S. Pat. No. 5,023,252.)

Optimal dosages to be administered may be readily determined by those skilled in the art, and will vary with the particular 5-HT_{1F} receptor agonist in use, the strength of the preparation, the mode of administration, and the advancement of the disease condition. Additional factors depending on the particular patient being treated will result in a need to adjust dosages, including patient age, weight, diet, and time of administration.

The term “therapeutically effective amount” as used herein refers to that amount of pharmaceutical agent that elicits in a tissue, system, animal or human, the biological or medicinal response that is being sought by a researcher, veterinarian, medical doctor or other clinician, which response includes alleviation of the symptoms of the disease being treated.

The term “subject,” as used herein refers to an animal, preferably a mammal, most preferably a human, who has been the object of treatment, observation or experiment.

Cell Transfections

Transient transfections of COS-7 cells with various plasmids were performed using the DEAE-Dextran method, which is well-known to those skilled in the art. Briefly, a plasmid comprising an expression vector for the receptor of interest was added to monolayers of COS-7 cells bathed in a DEAE-Dextran solution. In order to enhance the efficiency of transfection, dimethyl sulfoxide was typically also added, according to the method of Lopata (Lopata, et al., 1984). Cells were then grown under controlled conditions and used in experiments after about 72 hours.

Stable cell lines were obtained using means which are well-known in the art. For example, a suitable host cell was typically cotransfected, using the calcium phosphate technique, with a plasmid comprising an expression vector for the receptor of interest and a plasmid comprising a gene which allows selection of successfully transfected cells. Cells were then grown in a controlled environment and selected for expression of the receptor of interest. By continuing to grow and select cells, stable cell lines were obtained expressing the receptors described and used herein.

Binding Assays

The binding of a test compound to a receptor of interest was generally evaluated by competitive binding assays using membrane preparations derived from cells which expressed the receptor. First, conditions were determined which allowed measurement of the specific binding of a compound known to bind to the receptor. Then, the binding of the known compound to the receptor in membrane preparations was evaluated in the presence of several different concentrations of the test compound. Binding of the test compound to the receptor resulted in a reduction in the amount of the known compound which was bound to the receptor. A test compound having a high affinity for the receptor of interest would displace a given fraction of the bound known compound at a concentration lower than the concentration which would be required if the test compound had a low affinity for the receptor of interest.

This invention will be better understood from the Experimental Details which follow. However, one skilled in the art will readily appreciate that the specific methods and results discussed are merely illustrative of the invention as described more fully in the claims which follow thereafter.

Experimental Details

The following Experimental Details are set forth to aid in understanding the invention, and are not intended, and should not be construed, to limit in any way the invention set forth in the claims which follow thereafter.

5-HT_{1F}-Selective Compounds

Compound 1 is N-(N,N-Dimethyl-1,2,3,4-tetrahydro-2-aminodibenzofur-8-yl)-pyridine-4-carboxamide. The synthesis of this compound is described in Example 18 of U.S. Pat. No. 5,846,995, issued Dec. 8, 1998.

Compound 2 is (R)-(+)-6-(4-fluorobenzoyl)amino-2-(dimethyl)amino-1,2,3,4-tetrahydro-9H-carbazole. The synthesis of this compound is described in Example 114 of U.S. Pat. No. 5,814,653, issued Sep. 29, 1998.

Other 5-HT_{1F} Agonists

5-HT_{1F} agonists are well-known in the art. See, for example U.S. Pat. Nos. 5,521,196, 5,521,197, 5,721,252, 5,792,763, 5,814,653, 5,817,671, 5,846,995, 5,905,084, 5,708,008, 5,708,187, 5,814,653, 5,708,187, 5,919,936 and 5,874,427, the disclosures of which are hereby incorporated by reference in their entireties into this application.

1-((2S)-hydroxy-3-(naphth-2-yloxy)prop-1-yl)-4-hydroxy-4-(quinolin-3-yl)piperidine (Compound 3)

1-tert-butoxycarbonyl-4-piperidone:

A solution of 9.0 gm (61.5 mMol) 4-piperidone hydrochloride monohydrate in dioxane/water at 0° C. was treated sequentially with aqueous sodium carbonate and 14.4 gm (68 mMol) 2,2-dimethylpropanoic anhydride (BOC anhydride). The resultant slurry was stirred vigorously at room temperature for 18 hours. The reaction mixture was then concentrated under reduced pressure and the residue diluted with ethyl acetate. This mixture was treated with 1.5 M aqueous sodium hydrogen sulfate until the pH was about 2. The layers were separated and the remaining organics were washed with saturated aqueous sodium chloride, dried over sodium sulfate and concentrated under reduced pressure to give 9.8 gm (80%) of the title compound as a tan solid. Elemental Analysis: Calculated for: C₁₀H₁₇NO₃: Theory: C, 60.28; H, 8.60; N, 7.03. Found: C, 60.12; H, 8.54; N, 7.11. MS (m/e): 199 (M+)

4-(quinolin-3-yl)-4-hydroxypiperidine:

3-bromoquinoline is reacted with an alkyllithium, typically n-butyllithium or sec-butyllithium, at about −100° C. to about −78° C. for from 1 hour to about 4 hours in a suitable solvent, such as diethyl ether or tetrahydrofuran. To the quinoline-Li formed in this manner is added 1-tert-butoxycarbonyl-4-piperidone and the reaction is stirred from about 4 hours to about 24 hours at room temperature. The resultant alcohol is isolated by extractive workup and may be used as isolated for subsequent reactions or purified by chromatography if necessary. The alcohol is N-deprotected in a suitable solvent, typically dichloromethane, at room temperature for from about 4 hours to about 24 hours. Excess acid is neutralized with an appropriate base, typically sodium or potassium hydroxide, and the desired product is isolated by normal extractive work up. The product may be used as is or purified by chromatography if necessary or desired.

Beginning with 7.46 gm (35.9 mMol) 3-bromoquinoline, 7.25 gm (62%) N-tert-butoxycarbonyl-4-(quinolin-3-yl)-4-hydroxypiperidine were recovered as a light yellow solid by the procedure described above. Beginning with 1.5 gm (4.6 mMol) N-tert-butoxy-carbonyl-4-(quinolin-3-yl)-4-hydroxypiperidine, 0.645 gm (62%) of the title compound were recovered as a light tan solid by the procedure described above. MS (m/e): 228 (M⁺); Calculated for C₁₄H₁₆N₂O-0.25H₂O: Theory: C, 72.23; H, 7.14; N, 12.03. Found: C, 72.41; H, 7.12; N, 12.89.

(S)-glycidylnaphthyl-2-yl ether:

The aryl alcohol is deprotonated with a suitable base, typically sodium hydride, in a suitable solvent, typically dimethylformamide, at from about 0° C. to about room temperature. (The aryl alcohol is 2-hydroxynaphthalene.) The anion is then reacted with glycidyl-3-nitrobenzenesulfonate at room temperature for from about 1 hour to about 24 hours. The product is isolated by normal extractive workup and may be used as isolated for subsequent reactions, or purified by chromatography or crystallization if necessary or desired.

1-((2S)-hydroxy-3-(naphth-2-yloxy)prop-1-yl)-4-hydroxy-4-(quinolin-3-yl)piperidine (Compound 3):

A mixture of (S)-glycidylnaphth-2-yl ether and 4-hydroxy-4-(quinolin-3-yl)piperidine in 10 mL methanol was heated at reflux for 18 hours. The reaction mixture was then cooled to room temperature and partitioned between ethyl acetate and 2N sodium hydroxide. The phases were separated and the aqueous phase extracted well with ethyl acetate. The combined organic phases were washed with saturated aqueous sodium chloride, dried over sodium sulfate and concentrated under reduced pressure. The residue was subjected to flash silica gel chromatography, eluting with 25:1 dichloromethane:methanol. Fractions containing product were combined and concentrated under reduced pressure to provide the product.

Beginning with 0.132 gm (0.7 mMol) (S)-glycidylnaphth-2-yl ether and 0.150 gm (0.7 mMol) 4-hydroxy-4-(quinolin-3-yl)piperidine, 0.139 gm (49%) of the title compound were recovered as a white solid.

MS (m/e): 428 (M⁺); [α]_{D}²⁵ (methanol)=−5.814° Calculated for C₂₇H₂₈N₂O₃-0.25H O. Theory: C, 74.89; H, 6.63; N, 6.47. Found: C, 74.71; H, 6.67; N, 6.37.

Radioligand Binding Assays

The binding properties of compounds at different human receptors were determined in vitro using cultured cell lines that selectively express the receptor of interest. These cell lines were prepared by transfecting the cloned cDNA or cloned genomic DNA or constructs containing both genomic DNA and cDNA encoding the human receptors as follows:

Human 5HT_{1B}, 5HT_{1D}, 5HT_{1E}, 5HT_{1F}, and 5HT₇ Receptors:

The cell lysates of LM(tk-) clonal cell line stably transfected with the genes encoding each of these 5-HT receptor-subtypes were prepared as described above. Cell membranes were suspended in 50 mM Tris-HCl buffer (pH 7.4 at 37° C.) containing 10 mM MgCl₂, 0.2 mM EDTA, 10 μM pargyline, and 0.1% ascorbate. The affinities of compounds were determined in equilibrium competition binding assays by incubation for 30 minutes at 37° C. in the presence of 5 nM [³H]-serotonin. Nonspecific binding was determined in the presence of 10 μM serotonin. The bound radioligand was separated by filtration through GF/B filters using a cell harvester.

Human 5HT_{2A} Receptor: The coding sequence of the human 5HT_{2A} receptor was obtained from a human brain cortex cDNA library, and cloned into the cloning site of pCEXV-3 eukaryotic expression vector. This construct was transfected into COS-7 cells by the DEAE-dextran method. Cells were harvested after 72 hours and lysed by sonication in 5 mM Tris-HCl, 5 mM EDTA, pH 7.5. The cell lysates were centrifuged at 1000 rpm for 5 minutes at 4° C., and the supernatant was centrifuged at 30,000× g for 20 minutes at 4° C. The pellet was suspended in 50 mM Tris-HCl buffer (pH 7.7 at room temperature) containing 10 mM MgSO₄, 0.5 mM EDTA, and 0.1% ascorbate. The affinity of compounds at 5-HT_{2A} receptors were determined in equilibrium competition binding assays using [³H] ketanserin (1 nM). Nonspecific binding was defined by the addition of 10 μM mianserin. The bound radioligand was separated by filtration through GF/B filters using a cell harvester.

5-HT_{1A} Receptor: The cDNA corresponding to the 5-HT_{1A} receptor open reading frames and variable non-coding 5′- and 3′-regions, was cloned into the eukaryotic expression vector pCEXV-3. These constructs were transfected transiently into COS-7 cells by the DEAE-dextran method, and harvested after 72 hours. Radioligand binding assays were performed as described above for the 5-HT₂A receptor, except that ³H-8-OH-DPAT was used as the radioligand and nonspecific binding was determined by the addition of 10 μM mianserin.

Other 5-HT Receptors: The binding assays using 5-HT₄ were preformed according to the procedures described in U.S. Pat. No. 5,766,879, the disclosure of which is hereby incorporated by reference in its entirety into this application. Other serotonin receptor binding assays were preformed according to published methods: 5-HT_{2B}: Kursar et al., 1992; 5-HT_{2C}: Saltzman et al., 1991; 5-HT₃: Hoyer and Neijt, 1987; 5-HT_{5A}: Plassat et al., 1992; 5-HT_{5B}: Matthes et al., 1993; 5-HT₆: Kohen et al., 1996.

Other Receptors: Binding assays using the histamine H₁ and H₂; dopamine D1, D2, D3, and D5; and α_{1A}, α_{1B}, and α₂ adrenergic receptors may be carried out according to the procedures described in U.S. Pat. No. 5,780,485, the disclosure of which is hereby incorporated by reference in its entirety into this application. Binding studies for the β-adrenoceptor may be performed according to the method of Riva and Creese, 1989.

Results

The affinities of 5-HT_{1F}-selective compounds at various human serotonin receptor subtypes are shown below in Table 1.

In Vivo Model of the Micturition Reflex

The effects of compounds on the micturition reflex were assessed in the “distension-induced rhythmic contraction” (DIRC) model in rats, as described in previous publications (e.g. Maggi et al., 1987; Morikawa et al., 1992).

DIRC Model

Female Sprague Dawley rats weighing approximately 300 g were anesthetized with subcutaneous urethane (1.2 g/kg). The trachea was cannulated with PE240 tubing to provide a clear airway throughout the experiment. A midline abdominal incision was made and the left and right ureters were isolated. The ureters were ligated distally (to prevent escape of fluids from the bladder) and cannulated proximally with PE10 tubing. The incision was closed using 4-0 silk sutures, leaving the PE10 lines routed to the exterior for the elimination of urine. The bladder was cannulated via the transurethral route using PE50 tubing inserted 2.5 cm beyond the urethral opening. This cannula was secured to the tail using tape and connected to a pressure transducer. To prevent leakage from the bladder, the cannula was tied tightly to the exterior urethral opening using 4-0 silk.

To initiate the micturition reflex, the bladder was first emptied by applying pressure to the lower abdomen, and then filled with normal saline in 100 μl increments (maximum=2 ml) until spontaneous bladder contractions occurred (typically 20-40 mmHg at a rate of one contraction every 1 to 2 minutes. Once a regular rhythm was established, vehicle (saline) or 5-HT_{1F}-selective compounds were administered i.p. to explore their effects on bladder activity. In some instances, a compound known to affect the micturition reflex (e.g. the GABA_{B} receptor agonist APMPA [3-aminopropyl-2-methyl-phosphinic acid]) was given as a “positive control”.

Results

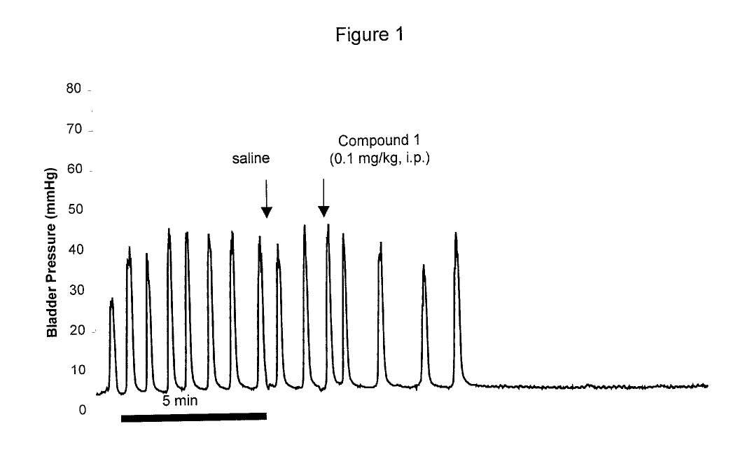

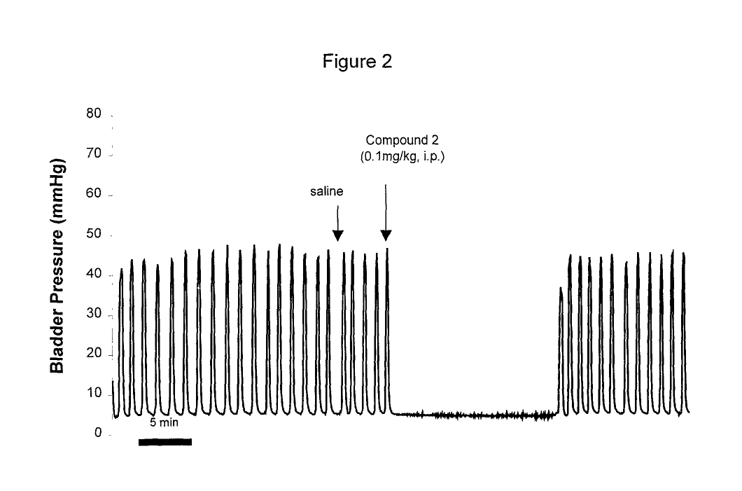

Distension-induced rhythmic contractions of the rat bladder were inhibited by the 5-HT_{1F}-selective agonists Compound 1 and Compound 2, (each at 0.1 mg/kg, i.p.). The effects of the agonists are shown in FIGS. 1 and 2.

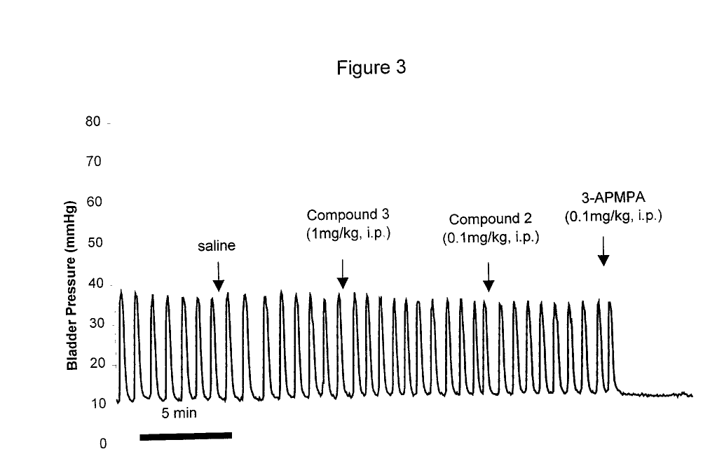

The prior administration of the 5-HT_{1F}-selective antagonist Compound 3 (1.0 mg/kg, i.p.) resulted in the complete blockade of the inhibitory effect of Compound 2 (FIG. 3). In contrast, Compound 3 failed to block the inhibitory effect of the GABA_{B}-selective agonist 3-APMPA (FIG. 3), demonstrating that the actions of Compounds 2 and 3 are specific to the 5-HT_{1F} receptor.

TABLE 1
Affinity of 5-HT_{1F}-selective compounds at various
human serotonin receptor subtypes (Kᵢ ± SEM (nM)).

| Receptor | Compound 1 | Compound 2 | Compound 3 |
|---|---|---|---|
| 5-HT_{1A} | 367 ± 42 | ND | 591 ± 143 |
| 5-HT_{1B} | 751 ± 68 | ND | 1285 ± 144 |
| 5-HT_{1D} | 1783 ± 87 | ND | 1534 ± 400 |
| 5-HT_{1E} | 2901 ± 288 | 461 ± 102 | 3670 ± 505 |
| 5-HT_{1F} | 7.11 ± 0.76 | 3.47 ± 0.08 | 4.31 ± 0.53 |
| 5-HT_{2A} | >5000 | ND | 377 ± 94 |
| 5-HT_{2B} | 4197 ± 219 | ND | 496 ± 36 |
| 5-HT_{2C} | 4760 ± 169 | ND | 1370 ± 341 |
| 5-HT₄ | 1873 ± 57 | ND | 405 ± 122 |
| 5-HT₆ | >5000 | ND | 4225 ± 399 |
| 5-HT₇ | >5000 | ND | 4406 ± 514 |

ND = Not Determined.

### REFERENCES

Andersson, K-E (1988) Current concepts in the treatment of disorders of micturition. Drugs 35: 477-494.

Ariens, E J et al. (1960) Receptor reserve and threshold phenomena. Arch. Int. Pharmacdyn. Ther. 127: 459-478.

DeGroat, W C (1998) Anatomy of the central neural pathways controlling the lower urinary tract. Eur. Urol. 34, suppl 1: 2-5.

DeGroat, W C and Steers, WD (1990) Autonomic regulation of the urinary bladder and sexual organs. In A D Loewy and K M Spyer eds., Central Regulation of Autonomic Functions, Oxford University Press, New York, pp. 310-333.

DeGroat, W C, Araki, I, Vizzard, M A, Yoshiyama, M, Yoshimura, N, Sugaya, K, Tai, C and Roppopo, J R (1998) Developmental and injury induced plasticity in the micturition reflex pathway. Behav. Brain Res. 92: 127-140.

Grant, T L, Ohnmacht, C J and Howe, B B (1994) Anilide tertiary carbinols: a novel series of K⁺ channel openers. Trends Pharmacol. Sci. 15: 402-404.

Hoyer, D and Neijt, H C (1987) Identification of serotonin 5-HT₃ recognition sites by radioligand binding in NG108-15 neuroblastoma-glioma cell. Eur. J. Pharmacol. 143: 191-192.

Kenakin, T P (1987) In: Pharmacological Analysis of Drug-Receptor Interaction. Raven Press, New York, pp. 190-192.

Kohen, R, Metcalf, M A, Khan, N, Druck, T, Huebner, K, Lachowicz, J E, Meltzer, H Y, Sibley, D R, Roth, B L and Hamblin, M W (1996) Cloning, characterization, and chromosomal localization of a human 5-HT6 serotonin receptor. J. Neurochem. 66: 47-56.

Kursar, J D, Nelson, D L, Wainscott, D B and Baez, M (1994) Molecular cloning, functional expression, and mRNA tissue distribution of the human 5-hydroxytryptamine2B receptor. Mol. Pharmacol. 46: 227-234.

Lopata, M A, Cleveland, D W, Sollner-Webb, B (1984) High level transient expression of a chloramphenicol acetyl transferase gene by DEAE-dextran mediated DNA transfection coupled with a dimethyl sulfoxide or glycerol shock treatment. Nucleic Acids Res. 12: 5707-5717.

Maggi, C A, Furio, M, Santicioli, P, Conte, B and Meli, A (1987) Spinal and supraspinal components of GABAergic inhibition of the micturition reflex in rats. J. Pharmacol. Exp. Ther. 240: 998-1005.

Matthes, H, Boschert, U, Amlaiky, N, Grailhe, R, Plassat, J L, Muscatelli, F, Mattei, M G and Hen, R (1993) Mouse 5-hydroxytryptamine5A and 5-hydroxytryptamine5B receptors define a new family of serotonin receptors: cloning, functional expression, and chromosomal localization. Mol. Pharmacol. 43: 313-319.

Morikawa, K, Hashimoto, S, Yamauchi, T, Kato, H, Ito and Y, Gomi, Y (1992) Inhibitory effect of inaperisone hydrochloride (inaperisone), a new centrally acting muscle relaxant, on the micturition reflex. Eur. J. Pharmacol. 213: 409-415.

Plassat, J L, Boschert, U, Amlaiky and Hen, R (1992) The mouse 5HT5 receptor reveals a remarkable heterogeneity within the 5HT1D receptor family. EMBO J. 11: 4779-4786.

Riva, M A and Creese, I (1989) Comparison of two putatively selective radioligands for labeling central nervous system beta-adrenergic receptors: inadequacy of [3H]dihydroalprenolol. Mol. Pharmacol. 36: 201-210.

Saltzman, A G, Morse, B, Whitman, M M, Ivanshchenko, Y, Jaye, M and Felder, S (1991) Cloning of the human serotonin 5-HT2 and 5-HT1C receptor subtypes. Biochem. Biophys. Res. Commun. 181: 1469-1478.

Swami, S K and Abrams P (1996) Urge Incontinence. Urodynamics II 23: 417-425.

Wein, A J (1987) Lower urinary tract function and pharmacologic management of lower urinary tract dysfunction. Current Medical Therapy for Urologic Disease 14: 273-296.

Yoshiyama, M, Roppolo, J R and DeGroat, W C (1995) Interactions between NMDA and AMPA/kinate receptors in the control of micturition in the rat. Eur. J. Pharmacol. 287: 73-78.

What is claimed is:

1. A method of treating urinary incontinence in a subject which comprises administering to the subject a therapeutically effective amount of a 5-HT_{1F} receptor agonist which activates the human 5-HT_{1F} receptor at least ten-fold more than it activates each of the human 5-HT_{1A}, 5-HT_{1D}, 5-HT_{2A}, 5-HT_{2C}, 5-HT₃, 5-HT₄, and 5-HT₇ receptors.

2. The method of claim 1, wherein the 5-HT_{1F} receptor agonist additionally activates the human 5-HT_{1F} receptor at least ten-fold more than it activates each of the 5-HT_{1B}, 5-HT_{1E}, 5-HT_{2B}, 5-HT_{5A}, 5-HT_{5B}, and 5-HT₆ receptors.

3. The method of claim 1, wherein the 5-HT_{1F} receptor agonist also activates the human 5-HT_{1F} receptor at least ten-fold more than it activates any human α₂ adrenoceptor or any human β adrenoceptor.

4. The method of claim 1, wherein the 5-HT_{1F} receptor agonist also activates the human 5-HT_{1F} receptor at least ten-fold more than it activates the human histamine HL and H₂ receptors.

5. The method of claim 1, wherein the 5-HT_{1F} receptor agonist also activates the human 5-HT_{1F} receptor at least ten-fold more than it activates the human dopamine D₁, D₂, D₃, and D₅ receptors.

6. The method of claim 1, wherein the 5-HT_{1F} receptor agonist also activates the human 5-HT_{1F} receptor at least ten-fold more than it activates the human α_{1A} adrenoceptor and the human α_{1B} adrenoceptor.

7. The method of claim 1, wherein the 5-HT_{1F} receptor agonist activates the human 5-HT_{1F} receptor at least 50-fold more than it activates each of the human 5-HT_{1A}, 5-HT_{1D}, 5-HT_{2A}, 5-HT_{2C}, 5-HT₃, 5-HT₄, and 5-HT₇ receptors.

8. The method of claim 7, wherein the 5-HT_{1F} receptor agonist additionally activates the human 5-HT_{1F} receptor at least 50-fold more than it activates each of the human 5-HT_{1B}, 5-HT_{1E}, 5-HT_{2B}, 5-HT_{5A}, 5-HT_{5B}, and 5-HT₆ receptors.

9. The method of claim 7, wherein the 5-HT_{1F} receptor agonist also activates the human 5-HT_{1F} receptor at least ten-fold more than it activates any human α₂ adrenoceptor or any human β adrenoceptor.

10. The method of claim 7, wherein the 5-HT_{1F} receptor agonist also activates the human 5-HT_{1F} receptor at least ten-fold more than it activates the human histamine H₁ and H₂ receptors.

11. The method of claim 7, wherein the 5-HT_{1F} receptor agonist also activates the human 5-HT_{1F} receptor at least ten-fold more than it activates the human dopamine D₁, D₂, D₃, and D₅ receptors.

12. The method of claim 7, wherein the 5-HT_{1F} receptor agonist also activates the human 5-HT_{1F} receptor at least ten-fold more than it activates the human α_{1A} adrenoceptor and the human α_{1B} adrenoceptor.

13. The method of claim 7, wherein the 5-HT_{1F} receptor agonist activates the human 5-HT_{1F} receptor at least 100-fold more than it activates each of the human 5-HT_{1A}, 5-HT_{1D}, 5-HT_{2A}, 5-HT_{2C}, 5-HT₃, 5-HT₄, and 5-HT₇ receptors.

14. The method of claim 13, wherein the 5-HT_{1F} receptor agonist additionally activates the human 5-HT_{1F} receptor at least 100-fold more than it activates each of the human 5-HT_{1B}, 5-HT_{1E}, 5-HT_{2B}, 5-HT_{5A}, 5-HT_{5B}, and 5-HT₆ receptors.

15. The method of claim 13, wherein the 5-HT_{1F} receptor agonist also activates the human 5-HT_{1F} receptor at least ten-fold more than it activates any human α₂ adrenoceptor or any human β adrenoceptor.

16. The method of claim 13, wherein the 5-HT_{1F} receptor agonist also activates the human 5-HT_{1F} receptor at least ten-fold more than it activates the human histamine H₁ and H₂ receptors.

17. The method of claim 13, wherein the 5-HT_{1F} receptor agonist also activates the human 5-HT_{1F} receptor at least ten-fold more than it activates the human dopamine D₁, D₂, D₃, and D₅ receptors.

18. The method of claim 13, wherein the 5-HT_{1F} receptor agonist also activates the human 5-HT_{1F} receptor at least ten-fold more than it activates the human α_{1A} adrenoceptor and the human α_{1B} adrenoceptor.

19. The method of claim 13, wherein the 5-HT_{1F} receptor agonist activates the human 5-HT_{1F} receptor at least 200-fold more than it activates each of the human 5-HT_{1A}, 5-HT_{1D}, 5-HT_{2A}, 5-HT_{2C}, 5-HT₃, 5-HT₄, and 5-HT₇ receptors.

20. The method of claim 19, wherein the 5-HT_{1F} receptor agonist additionally activates the human 5-HT_{1F} receptor at least 200-fold more than it activates each of the human 5-HT_{1B}, 5-HT_{1E}, 5-HT_{2B}, 5-HT_{5A}, 5-HT_{5B}, and 5-HT₆ receptors.

21. The method of claim 19, wherein the 5-HT_{1F} receptor agonist also activates the human 5-HT_{1F} receptor at least ten-fold more than it activates any human α₂ adrenoceptor or any human β adrenoceptor.

22. The method of claim 19, wherein the 5-HT_{1F} receptor agonist also activates the human 5-HT_{1F} receptor at least ten-fold more than it activates the human histamine H₁ and H₂ receptors.

23. The method of claim 19, wherein the 5-HT_{1F} receptor agonist also activates the human 5-HT_{1F} receptor at least ten-fold more than it activates the human dopamine D₁, D₂, D₃, and D₅ receptors.

24. The method of claim 19, wherein the 5-HT_{1F} receptor agonist also activates the human 5-HT_{1F} receptor at least ten-fold more than it activates the human α_{1A} adrenoceptor and the human α_{1B} adrenoceptor.
